(19) United States
(12) Patent Application Publication
(10) Pub. No.: US 2025/0352071 A1
(43) Pub. Date: Nov. 20, 2025

(54) MEDICAL DEVICE, ENDOSCOPE SYSTEM, CONTROL METHOD, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Nanako SATO, Sagamihara-shi (JP)

(73) Assignee: OLYMPUS MEDICAL SYSTEMS CORP., Tokyo (JP)

(21) Appl. No.: 19/288,273

(22) Filed: Aug. 1, 2025

Related U.S. Application Data
(63) Continuation of application No. PCT/JP2023/004402, filed on Feb. 9, 2023.

(51) Int. Cl. A61B 5/02; A61B 1/00; A61B 1/04; A61B 1/06; A61B 1/307; A61B 5/00; A61B 5/20

(52) U.S. Cl. CPC A61B 5/02; A61B 1/00009; A61B 1/00197; A61B 1/043; A61B 1/0638; A61B 1/0684; A61B 1/307; A61B 5/0071; A61B 5/0084; A61B 5/202

(57) ABSTRACT

A medical device includes a processor including hardware, the processor being configured to: acquire an imaging signal obtained by imaging an urinary bladder; generate a fluorescence image based on the imaging signal; identify a region constituted by pixels having a fluorescence intensity equal to or lower than a first fluorescence intensity from among pixels of the fluorescence image, as an insufficient heat denaturation region having an insufficient heat denaturation; identify a region constituted by pixels having a fluorescence intensity equal to or higher than a second fluorescence intensity from among the pixels of the fluorescence image, as an excessive heat denaturation region having an excessive heat denaturation; and output an output image on which the insufficient heat denaturation region and the excessive heat denaturation region are superimposed in an identifiable manner.

### CROSS REFERENCES TO RELATED APPLICATIONS

This application is a continuation of International Application No. PCT/JP2023/004402, filed on Feb. 9, 2023, the entire contents of which are incorporated herein by reference.

### BACKGROUND

### 1. Technical Field

The present disclosure relates to a medical device, an endoscope system, a control method, and a computer-readable recording medium.

### 2. Related Art

There is known a technique to visualize a heat denaturation state of biological tissue in treatment of the biological tissue with an energy device or the like (e.g. See WO 2020/054723 A1 and JP 2017-23604 A).

In techniques described in WO 2020/054723 A1 and JP 2017-23604 A, the heat denaturation state of biological tissue is visualized, on the basis of a captured image obtained by imaging fluorescence generated from the biological tissue by irradiating the biological tissue with excitation light. Specifically, in the technique described in WO 2020/054723 A1, a region, of all pixels of the captured image, having a fluorescence intensity higher than a preset fluorescence intensity is displayed as a region with high heat denaturation. Furthermore, in the technique described in JP 2017-23604 A, a region, of the captured image, having a fluorescence intensity lower than a preset fluorescence intensity is displayed as the region with high heat denaturation.

### SUMMARY

In some embodiments, a medical device includes a processor including hardware, the processor being configured to: acquire an imaging signal obtained by imaging an urinary bladder; generate a fluorescence image based on the imaging signal; identify a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder; identify a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and output an output image on which the insufficient heat denaturation region and the excessive heat denaturation region are superimposed in an identifiable manner.

In some embodiments, an endoscope system includes: a light source device configured to emit excitation light; an endoscope including an imaging element; and a medical device including a processor comprising hardware, the processor being configured to: acquire an imaging signal obtained by imaging an urinary bladder with the imaging element; generate a fluorescence image based on the imaging signal; identify a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder; identify a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and output an output image on which the insufficient heat denaturation region and the excessive heat denaturation region are superimposed in an identifiable manner.

In some embodiments, provided is a control method executed by a medical device. The method includes: acquiring an imaging signal obtained by imaging an urinary bladder with the imaging element; generating a fluorescence image based on the imaging signal; identifying a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder; identifying a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and outputting an output image on which the insufficient heat denaturation region and the excessive heat denaturation region are superimposed in an identifiable manner.

In some embodiments, provided is a non-transitory computer-readable recording medium with an executable program stored thereon. The program causes a medical device to execute: acquiring an imaging signal obtained by imaging an urinary bladder with the imaging element; generating a fluorescence image based on the imaging signal; identifying a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder; identifying a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and outputting an output image on which the insufficient heat denaturation region and the excessive heat denaturation region are superimposed in an identifiable manner.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to an embodiment;

FIG. 2 is a block diagram illustrating a functional configuration of a main portion of the endoscope system according to an embodiment;

FIG. 3 is a graph illustrating a wavelength characteristic of excitation light emitted from a second light source unit;

FIG. 4 is a graph illustrating a transmission characteristic of a cut filter;

FIG. 5 is a diagram illustrating an observation principle in a fluorescence observation mode;

FIG. 6 is a diagram illustrating an observation principle in a normal light observation mode;

FIG. 7 is a flowchart illustrating a control method performed by a control device;

FIG. 8 is a graph illustrating the control method;

FIG. 9 is a diagram illustrating the control method;

FIG. 10 is a diagram illustrating the control method;

FIG. 11 is a diagram illustrating the control method; and

FIG. 12 is a diagram illustrating the control method.

### DETAILED DESCRIPTION

Modes for carrying out the disclosure (hereinafter referred to as embodiments) will be described below with reference to the drawings. It should be understood that the disclosure is not limited to the embodiments described below. Furthermore, in illustration of the drawings, the same portions are denoted by the same reference numerals.

### Overall Configuration of Endoscope System

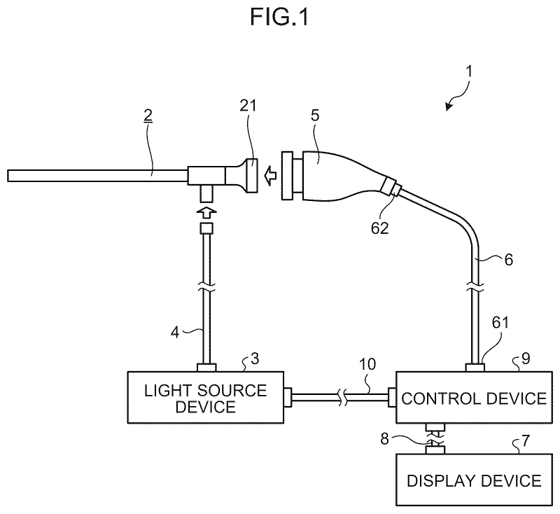

FIG. 1 is a diagram illustrating an overall configuration of an endoscope system 1 according to an embodiment.

The endoscope system 1 according to the present embodiment is an endoscope system that is used in holmium laser nucleation of the prostate (HoLEP) as surgical therapy for benign prostatic hyperplasia (BPH).

Specifically, in the holmium laser nucleation of the prostate is surgical therapy to apply a holmium: YAG laser to a boundary between inner gland and outer gland of enlarged prostate to enucleate the prostate.

As illustrated in FIG. 1, the endoscope system 1 includes an insertion section 2, a light source device 3, a light guide 4, a camera head 5, a first transmission cable 6, a display device 7, a second transmission cable 8, a control device 9, and a third transmission cable 10.

The insertion section 2 is rigid or at least partially flexible, has an elongated shape, and is inserted into a subject (the urinary bladder). In addition, the insertion section 2 is internally provided with an optical system such as a lens that forms a subject image.

The light source device 3 is connected to one end of the light guide 4 to supply illumination light for irradiation of the inside of the subject to the one end of the light guide 4 under the control of the control device 9. The light source device 3 is implemented by using one or more light sources of a light emitting diode (LED) light source, a xenon lamp, and a semiconductor laser element such as a laser diode (LD), a processor that is a processing device having hardware such as a field programmable gate array (FPGA) or a central processing unit (CPU), and a memory that is a temporary storage area used by the processor. Note that the light source device 3 and the control device 9 may be configured to communicate individually as illustrated in FIG. 1, or may be configured to be integrated with each other.

The one end of the light guide 4 is removably connected to the light source device 3 and the other end thereof is removably connected to the insertion section 2. Then, the light guide 4 guides the illumination light supplied from the light source device 3, from the one end to the other end and supplies the illumination light to the insertion section 2.

To the camera head 5, an eyepiece 21 of the insertion section 2 is removably connected. Under the control of the control device 9, the camera head 5 receives the subject image formed by the insertion section 2, performs photoelectric conversion to generate image data (RAW data), and outputs the image data to the control device 9 through the first transmission cable 6.

The insertion section 2 and the camera head 5 which are described above correspond to an endoscope.

The first transmission cable 6 has one end that is removably connected to the control device 9 through a video connector 61, and the other end that is removably connected to the camera head 5 through a camera head connector 62. Then the first transmission cable 6 transmits the image data output from the camera head 5 to the control device 9 and transmits setting data, power, and the like output from the control device 9, to the camera head 5. Here, the setting data is a control signal for controlling the camera head 5, a synchronization signal, a clock signal, and the like.

The display device 7 is constituted by a display monitor such as liquid crystal or organic electro luminescence (EL) display, and displays a display image based on image data subjected to image processing in the control device 9, and various information about the endoscope system 1, under the control of the control device 9.

The second transmission cable 8 has one end that is removably connected to the display device 7, and the other end that is removably connected to the control device 9. Then, the second transmission cable 8 transmits the image data subjected to image processing in the control device 9, to the display device 7.

The control device 9 corresponds to a medical device. The control device 9 is implemented by using a processor that is a processing device including hardware such as a graphics processing unit (GPU), FPGA, or CPU, and a memory that is a temporary storage area used by the processor. Then, the control device 9 controls the operations of the light source device 3, the camera head 5, and the display device 7 in an integrated manner, through the first to third transmission cables 6, 8, and 10, according to programs recorded in the memory. In addition, the control device 9 performs various image processing on the image data input through the first transmission cable 6, and outputs the image data to the second transmission cable 8.

The third transmission cable 10 has one end that is removably connected to the light source device 3, and the other end that is removably connected to the control device 9. The third transmission cable 10 transmits control data from the control device 9 to the light source device 3.

### Functional Configuration of Main Part of Endoscope System

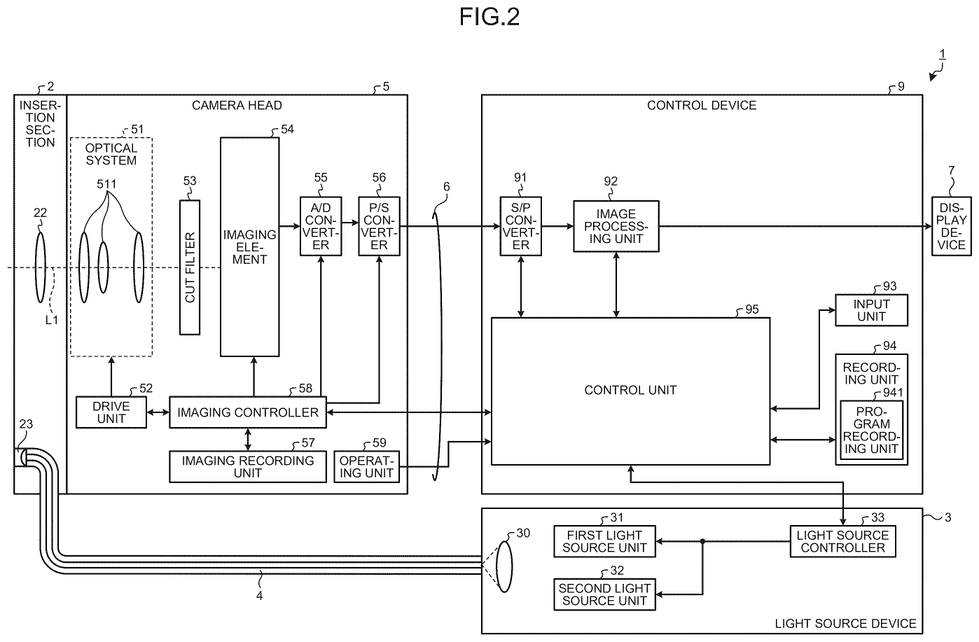

Next, a functional configuration of a main portion of the endoscope system 1 described above will be described. FIG. 2 is a block diagram illustrating a functional configuration of a main portion of the endoscope system 1.

Hereinafter, the insertion section 2, the light source device 3, the camera head 5, and the control device 9 will be described in this order.

### Configuration of Insertion Section

First, a configuration of the insertion section 2 will be described.

As illustrated in FIG. 2, the insertion section 2 includes an optical system 22 and an illumination optical system 23.

The optical system 22 is constituted by one or a plurality of lenses and the like, and condenses light such as reflected light reflected from a subject, return light from the subject, excitation light from the subject, and fluorescence emitted from the subject, forming the subject image.

The illumination optical system 23 is constituted by one or a plurality of lenses and the like, and irradiates the subject with the illumination light supplied from the light guide 4.

### Configuration of Light Source Device

Next, a configuration of the light source device 3 will be described.

As illustrated in FIG. 2, the light source device 3 includes a condenser lens 30, a first light source unit 31, a second light source unit 32, and a light source controller 33.

The condenser lens 30 condenses the light emitted from the first and second light source units 31 and 32 and outputs the light to the light guide 4.

Under the control of the light source controller 33, the first light source unit 31 emits white light (normal light) that is visible light to supply as the illumination light the white light to the light guide 4. The first light source unit 31 is configured using a collimating lens, a white LED lamp, a drive driver, and the like.

Note that the first light source unit 31 may use a red LED lamp, a green LED lamp, and a blue LED lamp to simultaneously emit light, supplying white light of visible light. Furthermore, the first light source unit 31 may include a halogen lamp, a xenon lamp, or the like.

Under the control of the light source controller 33, the second light source unit 32 emits excitation light having a predetermined wavelength band to supply as the illumination light the excitation light to the light guide 4.

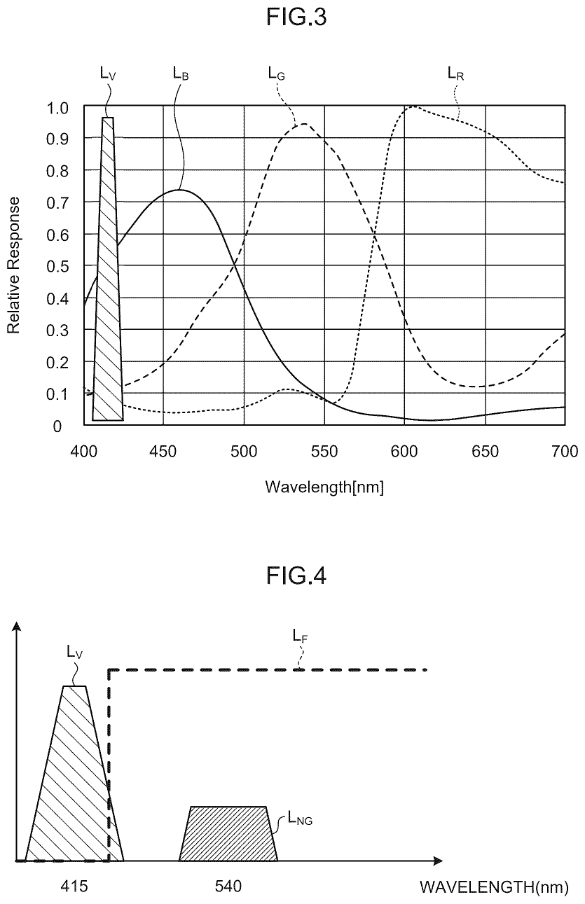

FIG. 3 is a graph illustrating a wavelength characteristic of the excitation light emitted from the second light source unit 32. In FIG. 3, the horizontal axis represents wavelength (nm) and the vertical axis represents wavelength characteristic. In FIG. 3, a curve L_{V} indicates a wavelength characteristic of the excitation light emitted from the second light source unit 32. Furthermore, in FIG. 3, a curve L_{B} indicates a blue wavelength band, a curve LG indicates a green wavelength band, and a curve LR indicates a red wavelength band.

Here, as illustrated in FIG. 3, the second light source unit 32 emits excitation light having a center wavelength (peak wavelength) of 415 nm and a wavelength band of 400 nm to 430 nm. The second light source unit 32 is configured using a collimating lens, a semiconductor laser such as a violet LD, a drive driver, and the like. The light source controller 33 is implemented by using a processor that is a processing device including hardware such as FPGA or CPU, and a memory that is a temporary storage area used by the processor. Then, the light source controller 33 controls the light emission timing, the light emission time, and the like of each of the first and second light source units 31 and 32, on the basis of the control data input from the control device 9.

### Configuration of Camera Head

Next, a configuration of the camera head 5 will be described.

As illustrated in FIG. 2, the camera head 5 includes an optical system 51, a drive unit 52, a cut filter 53, an imaging element 54, an A/D converter 55, a P/S converter 56, an imaging recording unit 57, an imaging controller 58, and an operating unit 59.

The optical system 51 forms the subject image focused by the optical system 22 of the insertion section 2, on a light receiving surface of the imaging element 54. The optical system 51 is configured using a plurality of lenses 511 (FIG. 2), and is configured to enable change of a focal length and a focal position. Specifically, the optical system 51 changes the focal length and the focal position by moving each of the plurality of lenses 511 on an optical axis L1 (FIG. 2) by the drive unit 52.

The drive unit 52 is configured using a motor such as a stepping motor, a DC motor, and a voice coil motor, and a transmission mechanism such as a gear that transmits rotation of the motor to the optical system 51. Then, the drive unit 52 moves the plurality of lenses 511 of the optical system 51 along the optical axis L1 under the control of the imaging controller 58.

The cut filter 53 is arranged on the optical axis L1, between the optical system 51 and the imaging element 54. The cut filter 53 blocks light having a predetermined wavelength band and transmits the other light.

FIG. 4 is a graph illustrating a transmission characteristic of the cut filter 53. Specifically, in FIG. 4, the horizontal axis represents wavelength (nm) and the vertical axis represents wavelength characteristic. Furthermore, in FIG. 4, a curve L_{E} indicates the transmission characteristic of the cut filter 53, and the curve L_{V} indicates the wavelength characteristic of the excitation light. Furthermore, in FIG. 4, a curve LNG indicates a wavelength characteristic of fluorescence generated by irradiating advanced glycation end products generated by laser irradiation (heat treatment) using an energy device for biological tissue, for example, a holmium: YAG laser, with excitation light.

Here, as illustrated in FIG. 4, the cut filter 53 partially blocks excitation light reflected from the biological tissue in an observation area, and transmits light having another wavelength band including a fluorescent component. More specifically, the cut filter 53 partially blocks light having a wavelength band on a short wavelength side of 400 nm to less than 430 nm including the excitation light, and transmits light having a wavelength band on a longer wavelength side than 430 nm, including the fluorescence generated by irradiating the advanced glycation end products generated by heat treatment with the excitation light.

The imaging element 54 is configured using a charge coupled device (CCD) or complementary metal oxide semiconductor (CMOS) image sensor in which any one of color filters constituting a Bayer array (RGGB) is arranged in each of a plurality of pixels arranged in a two-dimensional matrix. Then, under the control of the imaging controller 58, the imaging element 54 receives the subject image formed by the optical system 51 through the cut filter 53, generates the image data (RAW data) by photoelectric conversion, and outputs the image data to the A/D converter 55.

The A/D converter 55 is configured using an A/D conversion circuit or the like, performs A/D conversion processing on analog image data input from the imaging element 54 under the control of the imaging controller 58, and outputs the converted image data to the P/S converter 56.

The P/S converter 56 is configured using a P/S conversion circuit or the like, performs parallel/serial conversion of digital image data (corresponding to the captured image) input from the A/D converter 55 under the control of the imaging controller 58, and outputs the digital image data to the control device 9 through the first transmission cable 6.

Note that it may be configured that instead of the P/S converter 56, an E/O converter that converts image data into an optical signal may be provided and the image data may be output to the control device 9 by using the optical signal. Furthermore, it may be configured that the image data may be transmitted to the control device 9 by wireless communication such as Wireless Fidelity (Wi-Fi) (registered trademark).

The imaging recording unit 57 is constituted by a non-volatile memory or a volatile memory to record various information (e.g., pixel information of the imaging element 54 and a characteristic of the cut filter 53) about the camera head 5. Furthermore, the imaging recording unit 57 records various setting data and control parameters that are transmitted from the control device 9 through the first transmission cable 6.

The imaging controller 58 is implemented by using a timing generator (TG), a processor that is a processing device having hardware such as CPU, and a memory that is a temporary storage area used by the processor. Then, the imaging controller 58 controls the operation of each of the drive unit 52, the imaging element 54, the A/D converter 55, and the P/S converter 56, on the basis of the setting data received from the control device 9 through the first transmission cable 6.

The operating unit 59 is constituted by a button, a switch, and the like to receive a user operation by a user such as an operator and output an operation signal corresponding to the user operation to the control device 9. An example of the user operation includes an operation of switching an observation mode of the endoscope system 1 to a normal light observation mode or a fluorescence observation mode.

### Configuration of Control Device

Next, a configuration of the control device 9 will be described.

As illustrated in FIG. 2, the control device 9 includes an S/P converter 91, an image processing unit 92, an input unit 93, a recording unit 94, and a control unit 95.

Under the control of the control unit 95, the S/P converter 91 performs serial/parallel conversion on the image data received from the camera head 5 through the first transmission cable 6, and outputs the image data to the image processing unit 92.

Note that when the camera head 5 outputs the image data by using the optical signal, an O/E converter that converts an optical signal into an electric signal may be provided instead of the S/P converter 91. In addition, when the camera head 5 transmits the image data by wireless communication, a communication module that is configured to receive a wireless signal may be provided, instead of the S/P converter 91.

The image processing unit 92 corresponds to a processor. The image processing unit 92 is implemented by using a processor that is a processing device having hardware such as GPU or FPGA, and a memory that is a temporary storage area used by the processor. Then, under the control of the control unit 95, the image processing unit 92 performs predetermined image processing on the image data being the parallel data input from the S/P converter 91, and outputs the processed image data to the display device 7. Here, examples of the predetermined image processing include demosaic processing, white balance processing, gain adjustment processing, y correction processing, format conversion processing, and the like.

The input unit 93 is configured using a mouse, a foot switch, a keyboard, a button, a switch, a touch screen, and the like, receives a user operation by the user such as an operator, and outputs an operation signal corresponding to the user operation to the control unit 95.

The recording unit 94 is configured using a recording medium such as a volatile memory, a non-volatile memory, a solid state drive (SSD), a hard disk drive (HDD), or a memory card. Then, the recording unit 94 records data including various parameters and the like necessary for the operations of the endoscope system 1. Furthermore, the recording unit 94 includes a program recording unit 941 that records various programs for operating the endoscope system 1.

The control unit 95 is implemented by using a processor that is a processing device including hardware such as FPGA or CPU, and a memory that is a temporary storage area used by the processor. The control unit 95 controls the units constituting the endoscope system 1 in an integrated manner.

### Observation Principle in Observation Mode of Endoscope System

Next, observation principles in the observation modes of the endoscope system 1 will be described.

Hereinafter, the fluorescence observation mode and the normal light observation mode will be described in this order.

### Observation Principle in Fluorescence Observation Mode

First, an observation principle in the fluorescence observation mode will be described.

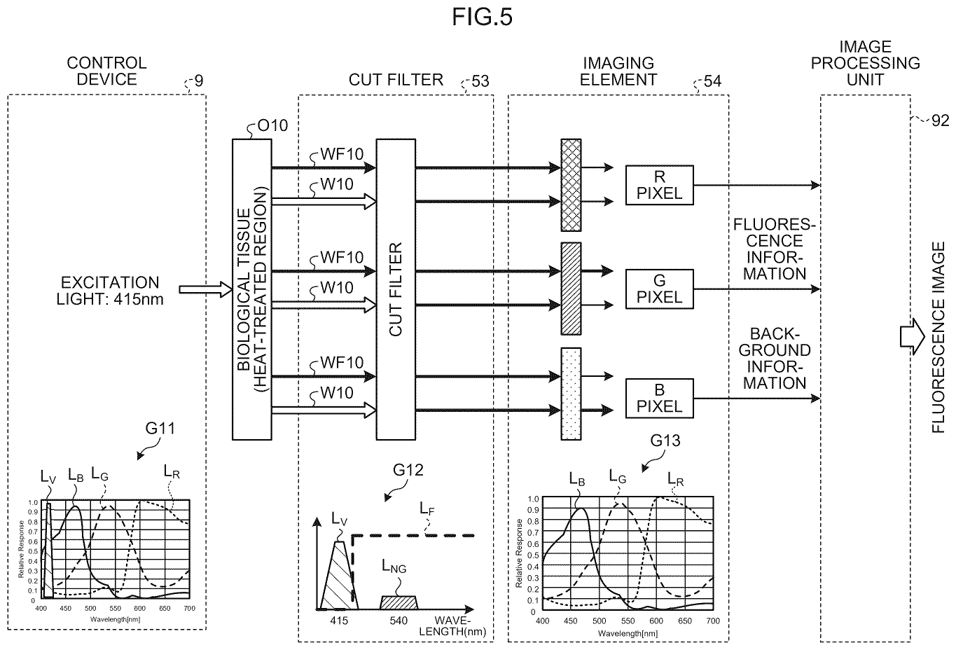

FIG. 5 is a diagram illustrating an observation principle in the fluorescence observation mode.

As illustrated in a graph G11 of FIG. 5, first, the light source device 3 causes the second light source unit 32 to emit light under the control of the control device 9 to irradiate biological tissue O10 (heat-treated region) subjected to laser irradiation (heat treatment) using the holmium: YAG laser, with the excitation light (center wavelength: 415 nm). In this case, as illustrated in a graph G12 of FIG. 5, reflected light (hereinafter, referred to as reflected light W10) including at least excitation light components and return light that are reflected from the biological tissue O10 (heat-treated region) is blocked by the cut filter 53 and the intensity thereof decreases, while some of components of the light on a wavelength side longer than the wavelength band to be blocked are applied to the imaging element 54 without decrease in the intensity of the light.

More specifically, as illustrated in the graph G12 of FIG. 5, the cut filter 53 blocks most of the reflected light W10 applied to a G pixel in the imaging element 54, having the wavelength band on the short wavelength side, including the wavelength band of the excitation light, and transmits light having the wavelength band on the longer wavelength side than the wavelength band to be blocked. In addition, as illustrated in the graph G12 of FIG. 5, the cut filter 53 transmits fluorescence WF10 obtained by autofluorescence of the advanced glycation end products in the biological tissue O10 (heat-treated region). Therefore, the reflected light W10 having a reduced intensity and the fluorescence WF10 are applied to each of an R pixel, the G pixel, and a B pixel in the imaging element 54.

Here, the G pixel in the imaging element 54 has a sensitivity to the fluorescence WF10. However, as indicated by the curve LNG of the fluorescence characteristic in the graph G12 of FIG. 5, the fluorescence has a minute reaction. Therefore, an output value corresponding to the fluorescence WF10 in the G pixel is a small value.

Thereafter, the image processing unit 92 acquires image data (RAW data) from the imaging element 54, performs image processing on the output values of the G pixel and the B pixel included in the image data, and generates a fluorescence image. In this configuration, the output value of the G pixel includes fluorescence information according to the fluorescence WF10 emitted from the heat-treated region. Furthermore, the output value of the B pixel includes background information from the biological tissue of the subject including the heat-treated region. Then, display of the fluorescence image on the display device 7 enables observation of the biological tissue (heat-treated region) thermally treated by the holmium: YAG laser.

### Observation Principle in Normal Light Observation Mode

Next, an observation principle in the normal light observation mode will be described.

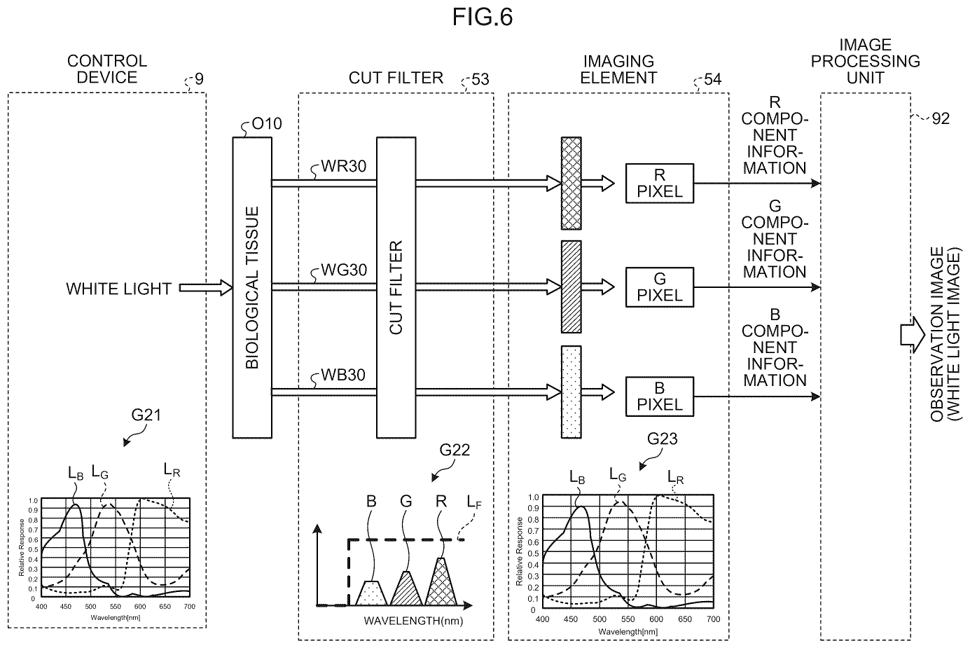

FIG. 6 is a diagram illustrating an observation principle in the normal light observation mode.

As illustrated in a graph G21 of FIG. 6, first, the light source device 3 causes the first light source unit 31 to emit light under the control of the control device 9 to irradiate the biological tissue O10 with the white light. In this configuration, reflected light and return light (hereinafter, described as reflected light WR30, WG30, and WB30) from the biological tissue O10 are partially blocked by the cut filter 53, and the rest is applied to the imaging element 54. Specifically, as illustrated in a graph G22 of FIG. 6, the cut filter 53 blocks reflected light having the wavelength band on the short wavelength side, including the wavelength band of the excitation light. Therefore, a component of light having the blue wavelength band applied to the B pixel in the imaging element 54 is smaller than that in a state where the cut filter 53 is not arranged.

Thereafter, the image processing unit 92 acquires image data (RAW data) from the imaging element 54, performs image processing on the output values of the R pixel, the G pixel, and the B pixel included in the image data, and generates an observation image (white light image). In this configuration, since a blue component included in the image data is smaller than that in a state where the cut filter 53 is not arranged, the image processing unit 92 performs white balance adjustment processing to adjust white balance so that proportions of a red component, a green component, and the blue component are constant. Then, display of the observation image (white light image) on the display device 7 enables observation of a natural observation image (white light image) even when the cut filter 53 is arranged.

### Control Method

Next, a control method performed by the control device 9 will be described.

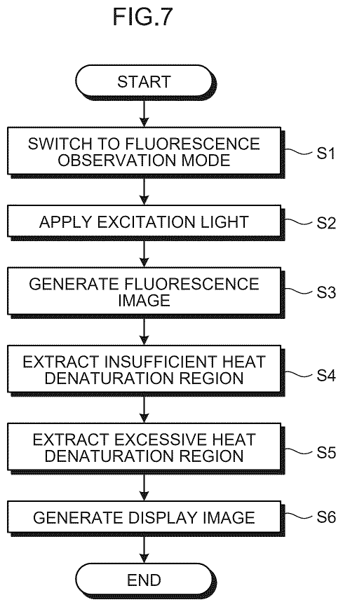

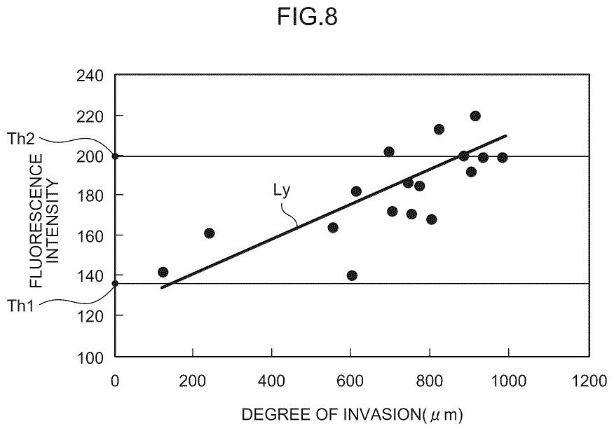

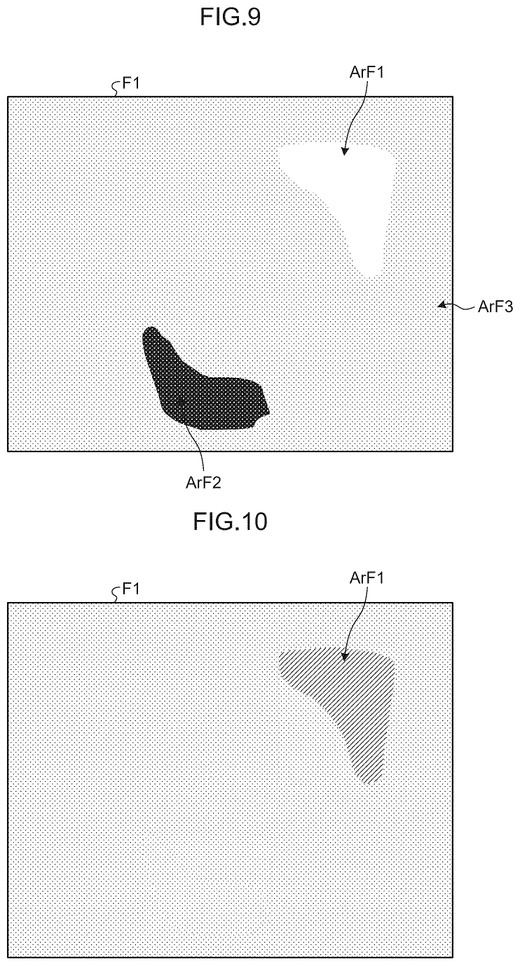

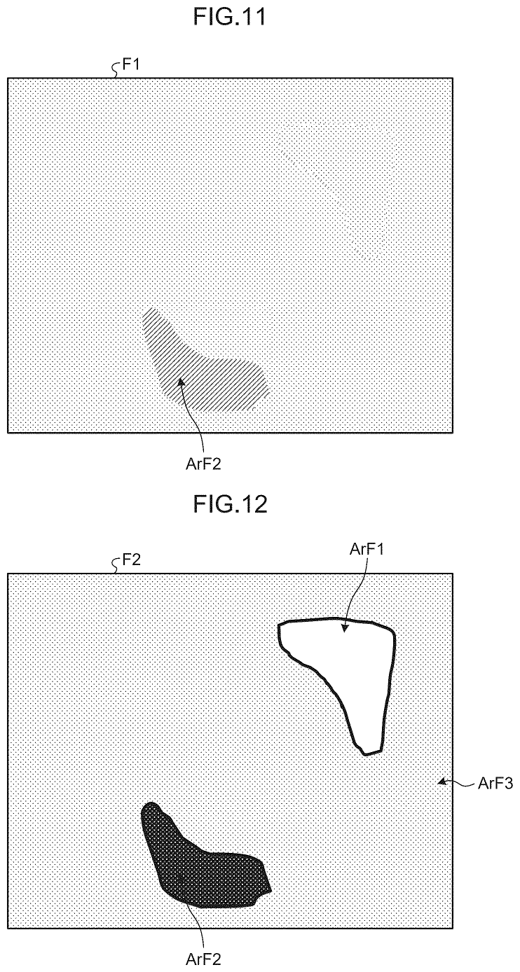

FIG. 7 is a flowchart illustrating a control method performed by the control device 9. FIGS. 8 to 12 are diagrams illustrating the control method. Specifically, FIG. 8 is a graph depicting a correlation (straight line Ly) between a fluorescence intensity of fluorescence obtained by autofluorescence of the advanced glycation end products in the biological tissue and a degree of invasion (depth and area) of the biological tissue by heat treatment. Note that, in FIG. 8, the vertical axis represents the fluorescence intensity, and the horizontal axis represents the degree of invasion of the biological tissue by heat treatment. FIG. 9 is a diagram illustrating a fluorescence image F1 generated in Step S3. FIG. 10 is a diagram corresponding to FIG. 9 and is a diagram illustrating Step S4. FIG. 11 is a diagram corresponding to FIG. 9 and is a diagram illustrating Step S5. FIG. 12 is a diagram corresponding to FIG. 9 and is a diagram illustrating a display image F2 generated in Step S6.

Note that hereinafter, the control method performed by the control device 9 during the holmium laser nucleation of the prostate will be described. In other words, the insertion section 2 is inserted into the subject (urinary bladder), and the observation area observed with the endoscope system 1 is the biological tissue (heat-treated region) on which heat treatment using the holmium: YAG laser is performed.

First, according to the operation of “switching the observation mode of the endoscope system 1 to the fluorescence observation mode” to the operating unit 59 by the user such as the operator, the control unit 95 switches the observation mode to the fluorescence observation mode (Step S1).

After Step S1, the control unit 95 controls the light source controller 33 to emit excitation light from the second light source unit 32 (Step S2).

After Step S2, the image processing unit 92 generates the fluorescence image on the basis of the image data generated by the imaging element 54 (Step S3).

Meanwhile, as depicted in FIG. 8, there is a correlation between the fluorescence intensity of the fluorescence obtained by autofluorescence of the advanced glycation end products in the biological tissue and the degree of invasion (degree of heat denaturation) of the biological tissue by heat treatment. Specifically, as indicated by the straight line Ly in FIG. 8, the fluorescence intensity increases as the degree of heat denaturation increases (as the degree of invasion of the biological tissue by heat treatment increases).

Here, in the fluorescence image F1 illustrated in FIG. 9, a region ArF1 filled with white is an insufficient heat denaturation region that is constituted by pixels having a fluorescence intensity equal to or lower than a first fluorescence intensity Th1 (FIG. 8), has a low degree of heat denaturation, and is insufficient in heat denaturation. In the insufficient heat denaturation region ArF1 configured as described above, insufficient heat denaturation results in insufficient hemostasis, and there may be bleeding after surgery. Furthermore, in the fluorescence image F1, a region ArF2 filled with black is an excessive heat denaturation region that is constituted by pixels having a fluorescence intensity equal to or higher than a second fluorescence intensity Th2 (FIG. 8), has a high degree of heat denaturation, and is excessive in heat denaturation. In the excessive heat denaturation region ArF2 configured as described above, excessive heat denaturation may lead to perforation after surgery. Furthermore, in the fluorescence image F1, a region ArF3 other than the regions ArF1 and ArF2 is an appropriate heat denaturation region that is constituted by pixels whose having a fluorescence intensity is larger than the first fluorescence intensity Th1 and smaller than the second fluorescence intensity Th2, and has an appropriate degree of heat denaturation.

Then, if the user such as the operator can recognize the insufficient heat denaturation region ArF1 and the excessive heat denaturation region ArF2, measures can be taken, reducing the risk of bleeding and perforation after surgery. However, even if the fluorescence image F1 is displayed on the display device 7, the fluorescence intensity displayed gradationally makes it difficult for the user such as the operator to recognize the regions ArF1 to ArF3 from the fluorescence image F1.

Therefore, in the present embodiment, Steps S4 to S6 described below are performed to generate a display image that enables identification of the regions ArF1 to ArF3.

Specifically, as illustrated in FIG. 10, the image processing unit 92 extracts the insufficient heat denaturation region ArF1 constituted by the pixels, from among all the pixels of the fluorescence image F1 generated in Step S3, having a fluorescence intensity equal to or lower than the first fluorescence intensity Th1 (Step S4).

Furthermore, as illustrated in FIG. 11, the image processing unit 92 extracts the excessive heat denaturation region ArF2 constituted by the pixels, from among all the pixels of the fluorescence image F1 generated in Step S3, having a fluorescence intensity equal to or higher than the second fluorescence intensity Th2 (Step S5).

Note that Steps S4 and S5 may be performed in the order of Steps S4 and S5, may be performed in the order of Steps S5 and S4, or may be performed substantially simultaneously in parallel.

Here, an example of the fluorescence intensity used in Steps S4 and S5 includes the output value of the G pixel in the imaging element 54, at least a g value of pixel values (r, g, b) of each pixel after demosaic processing on the image data acquired from the imaging element 54, a luminance value according to a Y signal (luminance signal), or the like.

Then, as illustrated in FIG. 12, the image processing unit 92 generates the display image F2 that enables identification of each of the regions ArF1 to ArF3 (Step S6).

Here, it is conceivable that “enables identification of each of the regions ArF1 to ArF3” means that the insufficient heat denaturation region ArF1, the excessive heat denaturation region ArF2, and the appropriate heat denaturation region ArF3 have colors different from each other. Note that the regions ArF1 to ArF3 may be entirely colored differently, or frame portions as outer edges of the regions ArF1 and ArF2 may be colored differently. FIG. 12 illustrates the regions ArF1 and ArF2 with the frame portions as the outer edges having different colors. In addition, each of the regions ArF1 to ArF3 may be identified by using an annotation (letters) in addition to the above-described color.

The present embodiment described above has the following effects.

In the control device 9 according to the present embodiment, the image processing unit 92 extracts a region constituted by the pixels, from among all the pixels of the captured image generated by the imaging element 54, having a fluorescence intensity equal to or lower than the first fluorescence intensity Th1, as the insufficient heat denaturation region ArF1 having insufficient heat denaturation. Then, the image processing unit 92 generates the display image F2 that enables identification of the insufficient heat denaturation region ArF1 of all the pixels of the captured image, from the other regions.

This configuration enables the user such as the operator to recognize the insufficient heat denaturation region ArF1 having insufficient heat denaturation of the biological tissue and insufficient hemostasis that may lead to bleeding after surgery, thus improving convenience.

Furthermore, in the control device 9 according to the present embodiment, the image processing unit 92 extracts a region constituted by pixels, of the captured image generated by the imaging element 54, having a fluorescence intensity equal to or higher than the second fluorescence intensity Th2 higher than the first fluorescence intensity Th1, as the excessive heat denaturation region ArF2. Then, the image processing unit 92 generates the display image F2 that enables identification of the excessive heat denaturation region ArF2 of all the pixels of the captured image, from the other regions.

This configuration enables the user such as the operator to recognize the excessive heat denaturation region ArF2 having excessive heat denaturation of the biological tissue that may lead to perforation after surgery, thus further improving convenience.

### OTHER EMBODIMENTS

The embodiment for carrying out the disclosure has been described above, but it should be understood that the disclosure is not limited only to the embodiment described above.

In the embodiment described above, the medical device has been mounted on the endoscope system used in the holmium laser nucleation of the prostate, but the disclosure is not limited thereto, and the medical device may be mounted on an endoscope system used in another procedure.

In the above embodiment, the medical device has been mounted on the endoscope system using a rigid endoscope, but the disclosure is not limited thereto, and the medical device may be mounted on an endoscope system using a flexible endoscope or an endoscope system using a medical surgical robot.

In the above embodiment, the display image F2 has been generated that enables identification of both the insufficient heat denaturation region ArF1 and the excessive heat denaturation region ArF2 of all the pixels of the captured image generated by the imaging element 54, from the other regions, but the disclosure is not limited thereto. For example, the display image F2 may be generated that enables identification of only the insufficient heat denaturation region ArF1 of all the pixels of the captured image, from the other regions.

In the above embodiment, the display image F2 has been generated that enables identification of the insufficient heat denaturation region ArF1 and the excessive heat denaturation region ArF2 of all the pixels of the fluorescence image F1, from the other regions, but the disclosure is not limited thereto. For example, the fluorescence observation mode and the normal light observation mode are switched alternately to generate the fluorescence image F1 and the observation image (white light image) in a time division manner. Then, a display image may be generated so as to enable identification of a region corresponding to the insufficient heat denaturation region ArF1 and a region corresponding to the excessive heat denaturation region ArF2 of all pixels of the observation image (white light image) generated at substantially the same timing as the fluorescence image F1, from the other regions.

In the above embodiment, the control unit 95 may have a function as a learning unit of a learning device. In this case, the control device 9 corresponds to the learning device.

Specifically, the control unit 95 uses, as input data, the fluorescence image obtained by imaging fluorescence generated from the biological tissue by irradiating the biological tissue with the excitation light, and uses training data using, as output data, information about the insufficient heat denaturation region, the excessive heat denaturation region, and the appropriate heat denaturation region that are extracted from the fluorescence image, for machine learning to generate a trained model. Note that the output data preferably includes at least information about the insufficient heat denaturation region, and may not include information about the excessive heat denaturation region and the appropriate heat denaturation region.

Here, the trained model includes a neural network in which each layer has one or a plurality of nodes. In addition, the type of machine learning is not particularly limited, but for example, it is preferable to prepare training data and learning data in which a plurality of fluorescence images of the subject are associated with at least information about the insufficient heat denaturation region extracted from the plurality of fluorescence images, input the training data and the learning data to a calculation model based on a multi-layer neural network to perform learning. Furthermore, for a machine learning method, for example, an approach based on a deep neural network (DNN) being the multi-layer neural network such as a convolutional neural network (CNN) or a 3D-CNN is used. Furthermore, an approach based on a recurrent neural network (RNN), long short-term memory units (LSTM) obtained by expanding RNN, or the like may be used, for the machine learning method. Note that these functions may be executed by a learning unit of a learning device different from the control device 9.

According to the medical device, the endoscope system, the control method, the control program, and a learning device according to the disclosure, convenience can be improved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising
a processor comprising hardware, the processor being configured to:
acquire an imaging signal obtained by imaging an urinary bladder;
generate a fluorescence image based on the imaging signal;
identify a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder;
identify a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and
output an output image on which the insufficient heat denaturation region and the excessive heat denaturation region are superimposed in an identifiable manner.

2. The medical device according to claim 1, wherein
the fluorescence image is an image that is obtained by capturing a fluorescence generated from advanced glycation end products generated by performing a heat treatment on a biological tissue of the urinary bladder.

3. The medical device according to claim 1, wherein
the processor is further configured to acquire a white light image obtained by imaging a biological tissue of the urinary bladder irradiated with white light, and
the output image is an image in which the insufficient heat denaturation region is displayed on the white light image so as to be identified from a region other than the insufficient heat denaturation region.

4. An endoscope system comprising:
a light source device configured to emit excitation light;
an endoscope including an imaging element; and
a medical device including a processor comprising hardware, the processor being configured to:
acquire an imaging signal obtained by imaging an urinary bladder with the imaging element;
generate a fluorescence image based on the imaging signal;
identify a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder;
identify a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and
output an output image on which the insufficient heat denaturation region and the excessive heat denaturation region are superimposed in an identifiable manner.

5. A control method executed by a medical device, the method comprising:
acquiring an imaging signal obtained by imaging an urinary bladder with the imaging element;
generating a fluorescence image based on the imaging signal;
identifying a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder;
identifying a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and
outputting an output image on which the insufficient heat denaturation region and the excessive heat denaturation region are superimposed in an identifiable manner.

6. A non-transitory computer-readable recording medium with an executable program stored thereon, the program causing a medical device to execute:
acquiring an imaging signal obtained by imaging an urinary bladder with the imaging element;
generating a fluorescence image based on the imaging signal;
identifying a region constituted by pixels having a fluorescence intensity equal to or lower than a first fluorescence intensity from among pixels of the fluorescence image, as an insufficient heat denaturation region having an insufficient heat denaturation, the insufficient heat denaturation region being a region where there is a possibility of bleeding after surgery due to a heat denaturation in the urinary bladder;
identifying a region constituted by pixels having a fluorescence intensity equal to or higher than a second fluorescence intensity from among the pixels of the fluorescence image, as an excessive heat denaturation region having an excessive heat denaturation, the second fluorescence intensity being larger than the first fluorescence intensity, the excessive heat denaturation region being a region where there is a possibility of perforation due to the heat denaturation in the urinary bladder; and
outputting an output image on which the insufficient heat denaturation region and the excessive heat denaturation region are superimposed in an identifiable manner.
